(19) United States
(12) Patent Application Publication
(10) Pub. No.: US 2023/0389875 A1
(43) Pub. Date: Dec. 7, 2023

(54) BREATHABLE AND SKIN-CONFORMAL ELECTRONICS WITH HYBRID INTEGRATION OF MICROFABRICATED MULTIFUNCTIONAL SENSORS AND KIRIGAMI-STRUCTURED NANOFIBROUS SUBSTRATES

(71) Applicant: The University of Hong Kong, Hong Kong (CN)

(72) Inventors: Lizhi XU, Changsha (CN); Hegeng LI, Wuhan (CN)

(21) Appl. No.: 18/302,865

(22) Filed: Apr. 19, 2023

Related U.S. Application Data
(60) Provisional application No. 63/365,740, filed on Jun. 2, 2022.

(51) Int. Cl. A61B 5/00; A61B 5/0205

(52) U.S. Cl. CPC A61B 5/6832; A61B 5/02055; A61B 2562/043; A61B 2562/12; A61B 2562/16

(57) ABSTRACT

The subject invention pertains to skin-integrated soft electronics achieving a multifunctional sensor platform combined with good breathability and conformability on the skin through a fabrication strategy that includes a hybrid integration of high-performance microfabricated sensors supported by nanofibrous soft substrates created with stamp-based transfer techniques combined with electrospinning. The resulting membrane devices exhibit tissue-like mechanical properties with high permeability for vapor transport. In addition, kirigami structures can be introduced into these membranes, providing high stretchability and 3D conformability for large-area integration on the skin. The multifunctional sensors array can provide spatiotemporal measurement of bioelectrical signals including temperature, skin hydration, and potentially many other physiological parameters. The robust performance and manufacturing scalability provided by these multifunctional skin electronics can create further opportunities for the development of advanced wearable systems.

### CROSS-REFERENCE TO RELATED APPLICATION

This application claims the benefit of U.S. Provisional Application Ser. No. 63/365,740, filed Jun. 2, 2022, which is hereby incorporated by reference in its entirety including any tables, figures, or drawings.

### BACKGROUND OF THE INVENTION

Skin-mounted electronic systems show great promise for the application in healthcare and human-machine interfaces.¹⁻⁶ These systems aim to monitor physiological parameters from the skin, which is the largest organ in the human body presenting abundant biophysical and biochemical signals. Ideal skin-electronics interfaces could possess several essential features: 1) structural flexibility or conformability sufficient to accommodate large areas of the skin contour; 2) arrays of high-performance sensors that enable spatiotemporal measurement in a reliable manner; and 3) biocompatibility and breathability of the devices that allow extended use in a wearable configuration.

Despite extensive effort over the past decade, achieving a combination of these essential attributes remains challenging. Biosensors built with traditional planar microfabrication techniques exhibit excellent electronic performances for physiological measurement,^{7,8} but they usually involve planar and rigid substrates which are not suited for large-area integration on the skin. Recent methods allowed for transfer printing of planar-fabricated electronics onto soft substrates.⁹⁻¹¹ However, such devices can be based on dense polymer films that hinder the transport of liquid and vapor. Their limited breathability represents a major concern for long-term application on the skin.

High performance, microfabricated sensors are based on rigid substrates, which cannot integrate over large area of a contoured skin surface. It is difficult to combine these sensing elements with soft, porous membranes into skin-compatible systems due to their distinct manufacturing conditions.

Alternatively, a variety of porous substrates involving woven fabrics,¹²⁻¹⁴ electrospun membranes,^{15,16} synthetic foams,^{17,18} plant-derived materials,^{19,20} etc., have been employed for the construction of wearable electronics with enhanced breathability. However, their integration with high-performance electronic sensors becomes difficult due to the incompatibility of processing conditions. In fact, electronic devices on porous substrates have been constructed with infiltrated conductive inks based on carbon nanotubes,^{21,22} metallic nanowires,^{23,24} liquid metals,^{25,26} conducting polymers^{27,28} or other components^{29,30}. The required printing processes create difficulties for the patterning of high-density sensors arrays. The low electrical conductivity from these inks also creates constraints for sensory performance. Furthermore, design of multifunctional system becomes challenging due to the limited materials options that can be directly processed on porous substrates. Selective encapsulation of the electronics without compromising the structural permeability represents another concern.

### BRIEF SUMMARY OF THE INVENTION

Skin-integrated soft electronics have attracted extensive research attention due to their potential utility for fitness monitoring, disease management, human—machine interfaces, and other applications. Although many materials and device components are explored for the construction of skin-integrated systems, achieving a multifunctional sensor platform combined with good breathability and conformability on the skin remains difficult. This challenge is partly due to the processing incompatibility between planar-fabricated microelectronics and biocompatible porous substrates.

Embodiments of the subject invention provide a fabrication strategy that can overcome this limitation, leading to large-area multifunctional skin electronics with breathability and conformability required for wearable applications. In certain embodiments, a hybrid integration of high-performance microfabricated sensors and nanofibrous soft substrates is made possible with stamp-based transferring techniques combined with electrospinning. The provided membrane devices exhibit tissue-like mechanical properties with high permeability for vapor transport. In addition, kirigami structures can be introduced into these membranes, providing high stretchability and 3D conformability for large-area integration on the skin. Embodiments comprising multifunctional sensor arrays allow for improved spatiotemporal measurement of bioelectrical signals, temperature, skin hydration, and many other physiological parameters. The robust performance and manufacturing scalability of the provided multifunctional skin electronics provides further opportunities for the development of advanced wearable systems, and such advancements are contemplated within the scope of the subject invention.

Embodiments of the subject invention provide an approach to the manufacturing of breathable and skin-conformal electronics that can overcome the limitations described above. In specific embodiments, a hybrid integration of high-performance sensors and breathable nanofibrous substrates can be provided with stamp-based transferring techniques followed by electrospinning. This manufacturing approach mitigates the processing incompatibility between planar microelectronic devices and porous substrates, leading to systems with excellent sensory performance in combination with breathability for long-term wearing on the skin. Furthermore, kirigami-inspired structures can be introduced in these membrane devices, allowing for large-area integration on the skin with conformal contact. Embodiments including multifunctional sensor arrays (e.g., an array of sensors capable of measuring temperature, electrophysiological signals, skin hydration, or other parameters) enable continuous measurement of body temperature, electrocardiogram (ECG), skin hydration, electromyogram (EMG), and potentially other physiological parameters (e.g., mechanical deformation, tissue stiffness, pressure, oxygenation, blood flow rate, biochemical species, etc.). The robust performance and manufacturing scalability provided by certain embodiments of electronics-skin interfaces can create advanced routes for wearable health monitoring, human-robot interaction, virtual/augmented reality, and many other applications.

Many devices described in related art are based on a dense substrate. Their breathability can be insufficient for wearable applications. Embodiments of the subject invention enable integration of high-performance electronic devices on nanoporous substrates (e.g., in one embodiment, pore size can be <10 micron, overall porosity >50%), which offer better breathability for skin-integration. Embodiments provide novel design in the fabrication process that allows hybrid integration of microelectronics and biocompatible porous substrates. The resulting systems have better breathability and allow for large-area integration on the skin for long term use.

The devices described in related literature are based on thick fibrous substrates infiltrated with conductive material, which work as electrodes only and cannot expand with other functions. Embodiments of the subject invention provide diverse integration of microfabricated high-performance sensors on nanoporous substrates, which offer better functionality (e.g., sensors for temperature, strain, hydration, electrophysiological parameters, and other sensory functions involving oxygenation, biochemical species, blood flow rate, tissue stiffness, etc.) for wearable applications.

Certain embodiments provide novel fabrication processes allowing for hybrid integration of planar-fabricated microelectronics and biocompatible porous substrates. Embodiments enable multifunctional sensors array for diverse modes of physiological monitoring on the skin.

Embodiments provide a novel manufacturing process that allows for transferring of microfabricated electronics onto soft nanofibrous membranes. This process mitigates the processing incompatibility between planar microelectronic devices and porous substrates, leading to systems with excellent sensory performance in combination with breathability and conformability for long-term wearing on the skin. Embodiments are related to a new product and the related process.

Embodiments of the device format and the manufacturing process are novel. Specifically, the devices can involve both planar fabricated multifunctional sensors and nanofibrous substrates, which has not been achieved before. The manufacturing process can involve a novel combination of stamp-based transfer techniques and electrospinning processes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates breathable and conformal electronic membranes (BCEMs) according to selected embodiments of the subject invention. (a) Schematics of the fabrication process illustrating stamp-based transferring techniques, electrospinning, and kirigami patterning. (b) A BCEM laminated on the back of a volunteer showing large-area skin integration. Scale bar equals cm. (c) Photographs showing serpentine electronics on top of the nanofibrous substrate (left) and in conformal contact (right) with the skin surface. Scale bar equals 10 mm. (d) A scanning electron microscopy (SEM) image showing the permeable nanofibrous structure of a BCEM. Scale bar equals 5 (e) Optical microscope images of various microsensors built in a BCEM. Scale bar equals 2 mm.

FIG. 2 illustrates mechanical behaviors of BCEMs according to selected embodiments of the subject invention. (a) Characterization of interfacial toughness between microfabricated polyimide (PI) components and styrene-ethylene-butylene-styrene (SEBS) substrate. (b) Stress-strain curve of a BCEM under tension. (c) Resistance change of interconnects in a BCEM as a function of tensile strain, as compared with a similar membrane without kirigami patterning. (d-e) Resistance change of interconnects in a BCEM with torsion up to 720° (d), or 10,000 cycles of 80% elongation (e). (f) A variation of kirigami design leading to higher stretchability for the electrical interconnect. (g) Finite element analysis (FEA) on the stress distribution in a BCEM under 25% of elongation, as compared with a device without kirigami cuts.

FIG. 3 illustrates breathability and biocompatibility of BCEMs according to selected embodiments of the subject invention. (a) Water vapor transmission as a function of time for dense SEBS film, nanofibrous SEBS membrane, polydimethylsiloxane (PDMS) film, and blank control. (b) A water droplet on a BCEM, showing the hydrophobicity provided by nanofibrous SEBS. Scale bar: 1 mm. (c) A fluorescent image of fibroblasts cultured on a BCEM showing good cell viability and normal morphology. Scale bar: 200 μm. (d) MTT (3-[4,5-dimethylthiazol-2-yl]-2,5 diphenyl tetrazolium bromide) assay showing cell proliferation on a BCEM over 3 days of incubation. (e) A series of photographs showing (left) three different samples laminated on the forearm, and infrared thermal images before (middle) and after (right) the running exercise, demonstrating the breathability of a nanofibrous SEBS as compared with dense SEBS and PDMS film.

FIG. 4 illustrates multi-modal physiological sensing with BCEMs according to selected embodiments of the subject invention. (a) A schematic showing the measurement scheme. (b) Comparison of ECG signals detected by commercial gel electrodes (top) and BCEM (bottom). (c) Temperature variation associated with cold compress applied on the skin, measured with a BCEM. (d) Electrical impedance as a function of skin hydration, measured with coaxial electrodes on a BCEM under various frequencies. (e) Decay of skin hydration after the application of moisturizing lotion, measured with both BCEM sensor and commercial sensor.

FIG. 5 illustrates EMG recordings and gesture recognition with BCEMs according to selected embodiments of the subject invention. (a) EMG signals arising from a pattern of hand motion. (b) The locations of three pairs of EMG electrodes mounted on the forearm, forming three independent channels for data acquisition. (c-d) Photographs of 5 different gestures (c), and the distinct patterns recorded by the three EMG channels (d).

FIG. 6 is a photograph of multifunctional sensors array fabricated on a 4-inch silicon wafer according to a selected embodiment of the subject invention.

FIG. 7 illustrates a microfabrication process on a 4-inch silicon wafer according to selected embodiments of the subject invention. (a) Spin coating PMMA and PI on 4-inch silicon wafer. (b and c) Photolithography and wet etching for the Pt metallization (b) and 2n d metallization (c), respectively. (d) Coating of the top encapsulation PI. (e) Overall pattern defined by RIE. (f) Dissolving PMMA in acetone.

FIG. 8 shows two photographs of a BCEM in the undeformed state (a) and under stretching (b), according to selected embodiments of the subject invention.

FIG. 9 shows three photographs of a BCEM conforming to the dynamic 3D surface of the skin, according to selected embodiments of the subject invention.

FIG. 10 illustrates Kirigami designs with various stretchability according to selected embodiments of the subject invention. Units for x, y, and L_{c} are in mm for this embodiment. The specific embodiment illustrated comprises an offset, regular, rectilinear array of long, narrow, aligned cuts, wherein each cut is aligned with the direction of a row in the array, and every other row of the array has offset columns such that the gap between cuts in one row is aligned with the center of a cut in adjacent rows. In this embodiment, the cuts in even numbered rows are aligned to form a first set of columns and the cuts in odd numbered rows are aligned to form a second set of columns offset from the first set of columns.

FIG. 11 illustrates tensile stress-strain curves for continuous SEBS (a) and Nanofibrous SEBS (b) according to selected embodiments of the subject invention.

FIG. 12 shows photographs of a BCEM subject to elongation according to an embodiment of the subject invention.

FIG. 13 shows a water droplet on a dense SEBS sheet according to an embodiment of the subject invention, showing a contact angle of 101°.

FIG. 14 illustrates the design of multifunctional sensors array for a BCEM according to an embodiment of the subject invention. (a) The overview of device. (b) Unipolar electrode. (c) Temperature sensor. (d) Electrodes for optoelectronics. (e) Hydration sensor.

FIG. 15 depicts an electrical impedance between skin and the electrodes, measured with a BCEM electrode according to an embodiment of the subject invention and a commercial Ag/AgCl electrode.

FIG. 16 shows ECG signals recorded by BCEMs before doing exercise (a), after one kilometer of running (b), and (c) continuous recording of ECG signals, showing the stability of the measurement with BCEM according to an embodiment of the subject invention.

FIG. 17 shows a calibration of the temperature sensor based on the temperature coefficient of resistance and hydration sensor based on the hydration curve of impedance according to an embodiment of the subject invention.

FIG. 18 shows a global model of finite element analysis (FEA) and mesh generation for a system according to an embodiment of the subject invention.

FIG. 19 shows a stress distribution of kirigami-structured substrate (a) and continuous substrate (b) in FEA for a system according to an embodiment of the subject invention.

### DETAILED DISCLOSURE OF THE INVENTION

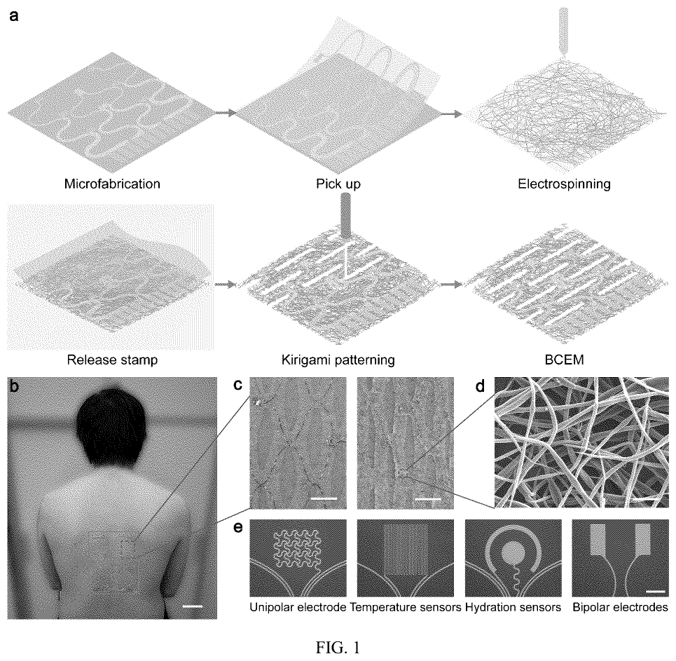

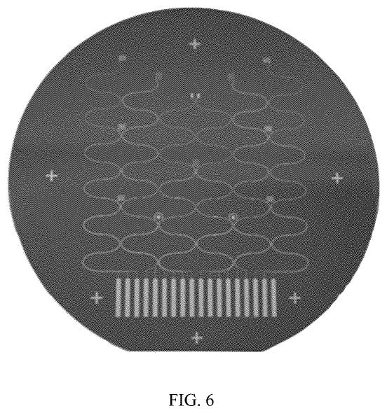

Construction of breathable and conformal electronic membranes (BCEMs) can start with fabrication of electronic components with stretchable serpentine patterns on a planar wafer (e.g., FIG. 1(a) and FIG. 6). This process benefits from established techniques involving thin-film deposition, photolithography, etching, etc., which can generate a variety of high-performance electronics based on metals, semiconductors, and polymers. The active device components are selectively encapsulated with a polyimide (PI) layer (e.g., about 5 μm in thickness) to inhibit crosstalk between sensors or current leakage. Selective encapsulation in accordance with various embodiments of the subject invention can include various encapsulation materials (e.g., PLGA, PLA, silk, or other materials which are bio-degradable). Selective encapsulation can include single layer or multi-layer constructs comprising electronic components with single layer or multi-layer encapsulation. Selective encapsulation can include various coverages, including for example coverage of only specified components, regions, or areas resulting in, for example, coverage of less than 10% of the surface area (e.g., measured in an undeformed, partially deformed, or deformed state; and measured either before or after application of surface modifications, such as trimming, shaping, or kirigami cutting), alternatively about 10, 20, 30, 40, 50, 60, 70, 80, 90, or more than 90% coverage, including increments, combinations, and ranges of any of the foregoing. Selective encapsulation can include various thicknesses, including for example one or more encapsulation layers about 5 μm thick, alternatively less than 1, about 1, 2, 3, 4, 5, 6, 7, 8, 9, 10, or more than 10 μm thick, including increments, combinations, and ranges of any of the foregoing.

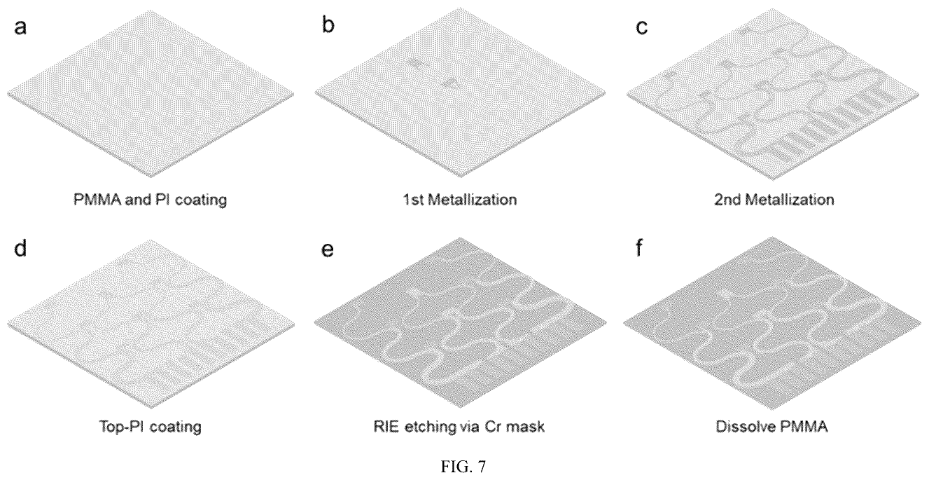

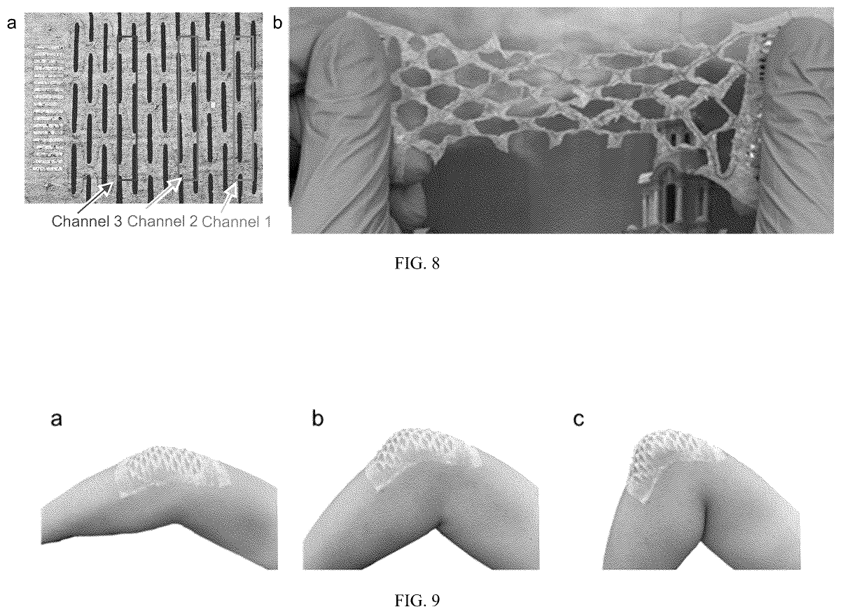

A sacrificial layer (e.g., based on poly(methyl methacrylate) (PMMA), metals dissolvable in acids, water-soluble salt and organics, or other dissolvable components, or coating-solvent systems) can be placed between the electronic components and the planar substrate, allowing for release of the serpentine devices from the handling wafer after dissolving the sacrificial layer (e.g., dissolving PMMA in acetone.) Subsequently, the electronics can be picked up with a stamp (e.g., water-soluble tape, or any tapes or rubber stamps that allow for subsequent release of the device) since the interaction between the device and the wafer is sufficiently weak after the dissolution of the sacrificial layer (FIG. 7). In the next step, polymer nanofibers (e.g., those from styrene-ethylene-butylene-styrene, or SEBS, or any polymer that can be electrospun such as PLGA, PLA, PVA, PU, etc.) can be electrospun on top of stamp-supported devices, which serves as a porous substrate with desired flexibility and mass permeability. Other polymers (e.g., polyurethane or poly (lactic-co-glycolic acid)) that are suitable for electrospinning are also potential candidates for the construction of BCEMs. Indeed, this process can be highly scalable since multiple device-on-stamps can be combined into a continuous membrane with roll-to-roll processing for electrospinning. In addition, variation of substrate materials and tuning of their structural parameters can be achieved during the electrospinning process. Dissolving the water-soluble tapes can yield freestanding membrane devices, which can be further processed with laser cutting to incorporate kirigami structures. Here, the periodic and alternating cuts can advantageously endow high stretchability of the structures (e.g., stretching above 80%, alternatively above 100%, above 120%, above 200%, above 250%, above 300%, above 350%, above 370%, or above 400% of the unstretched BCEM in one or more dimensions, including increments, ranges, and combinations thereof) as well as their conformability (e.g., conforming to an anatomical region such as the skin on the outside of a human elbow through a range of motion including no flexion, partial flexion, and full flexion, following application in a state of no flexion) on 3D curved surfaces (e.g., see FIGS. 8-9).^{31,32} Round edges in the kirigami cuts can be designed and implemented during the laser cutting process to mitigate stress concentration during deformation (FIG. 8).

FIG. 1(b) shows an embodiment of a BCEM conformally laminated on the back of a healthy volunteer, covering about 300 cm² of the skin surface, and enlarged image (FIG. 1(c) left) shows serpentine electronics integrated on the BCEM. The tissue-like nanofibrous substrate affords conformal contact and adequate adhesion based on van der Waals interactions.^{[33]} The dimension of the sensors array exceeded the size limit of individual handling wafer (4 inches in diameter), demonstrating the scalability of the integration process. In certain embodiments, the intrinsic flexibility of the SEBS nanofibers couples with the enhanced deformability arising from kirigami structures, which can provide mechanical support for the intimate contact between sensors and the contoured skin surface (FIG. 1(c) right). In addition, the permeable nanofiber network (FIG. 1(d)) can provide good breathability of membrane device. The serpentine configuration of the electronic components minimizes the mechanical restriction imposed to the membrane structures. Embodiments can incorporate microfabricated bipolar electrodes, unipolar electrodes, temperature sensors, and hydration sensors into the device platform (FIG. 1(e)). Embodiments can also provide many other devices (e.g., photodiodes, LEDs, strain sensors, pressure sensors, ultrasonic transducers, bioamplifiers, biochemical sensors, glucose sensors, sweat sensors, etc.) involving inorganic semiconductors or biochemical reagent that can be included during the fabrication process for extended functions (e.g., wearable healthcare system, or smart electronic wound dressing with or without biodegradable components).

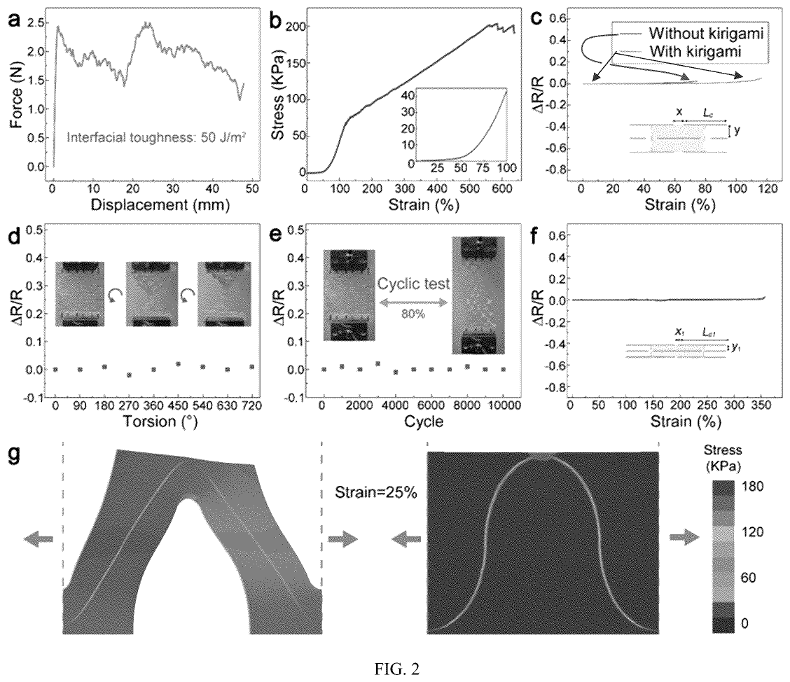

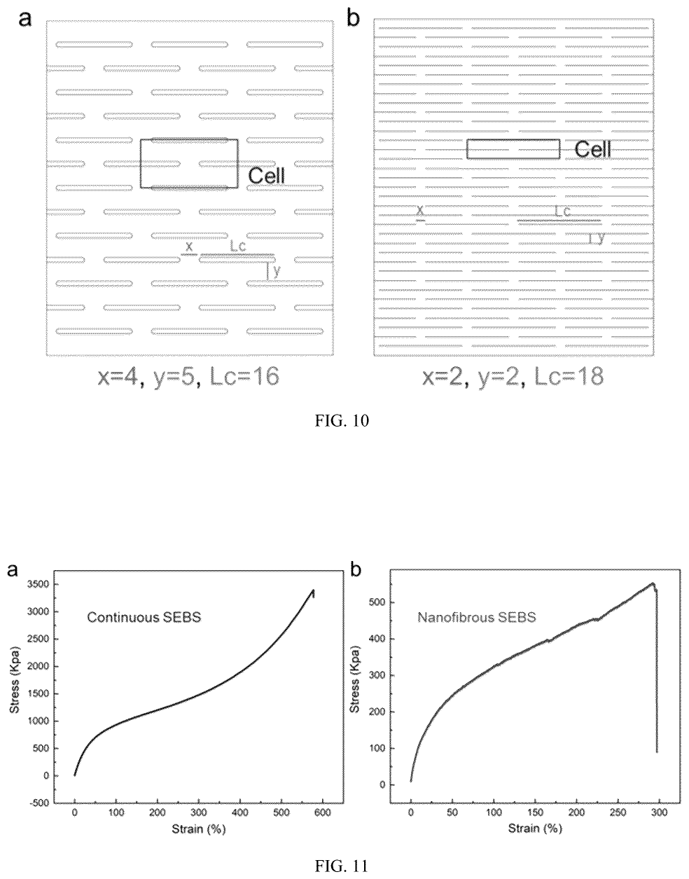

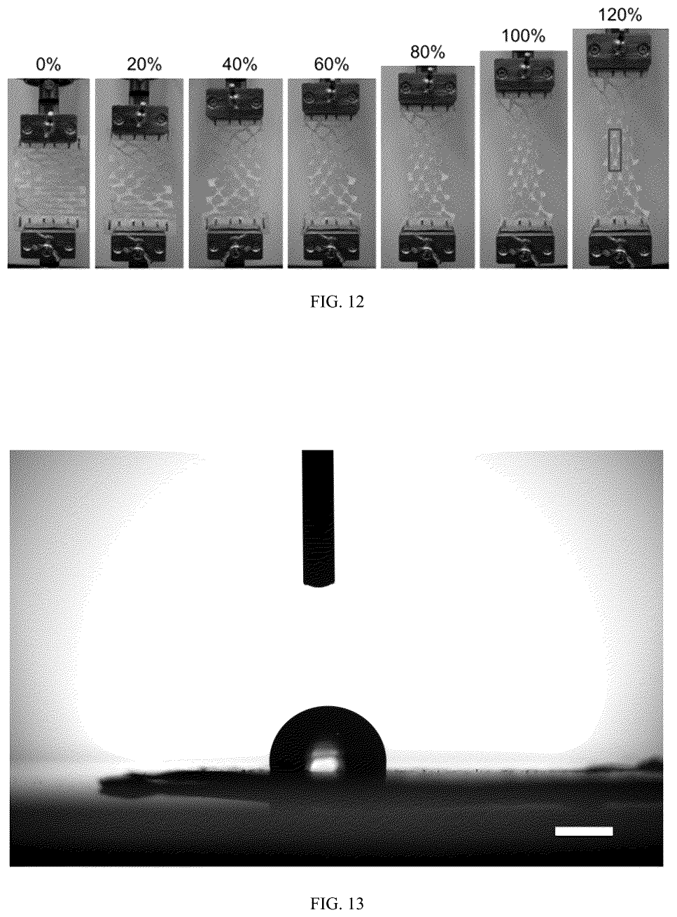

The inventors have systematically characterized the behaviors of certain embodiments of the BCEMs under mechanical deformation to evaluate their robustness during use. The physical integrity of certain embodiments can provide sufficient adhesion (e.g., sufficient to withstand stresses encountered in manufacturing, distribution, storage, packaging, unpackaging, preparation, application, and/or usage; as can be measured by adhesion or pull-apart tests known in the art including but not limited to ASTM D3330 or ASTM F2258) between SEBS and PI layers, with an interfacial toughness of about 50 J/m² (FIG. 2(a)). Furthermore, the kirigami patterns introduced in the membrane can accommodate large deformation without causing excessive stress which can cause interfacial delamination. FIG. 2(b) illustrate the strain-stress curve of a BCEM (also in FIG. 10(a)). The device possesses ultra-low modulus of 5 kPa below 50% of tensile strain due to the stretchability of kirigami structures, which matches the regime of natural motion of human skin.^{33,34} Under high elongation, the mechanical behavior of certain embodiments of BCEMs is associated with the intrinsic deformation of the nanofibrous network, which is consistent with the responses of nanofibrous SEBS membranes without kirigami patterning (FIG. 11). The combination of kirigami structures and the serpentine electronic components confers deformation-invariant electrical performance to certain embodiments (FIG. 2(c-e)). As a BCEM was stretched uniaxially from 0% to 120%, there was negligible change in resistance (ΔR/R₀) for the serpentine interconnects even with severe distortion (FIG. 12). On the other hand, serpentine interconnects bonded to a continuous membrane without kirigami cuts experienced failure at a much lower tensile strain of 70%. The deformation-invariant electrical properties were also observed when the device was twisted by 720° (FIG. 2d) or loaded with 10,000 cycles of 80% elongation (FIG. 2(e)).

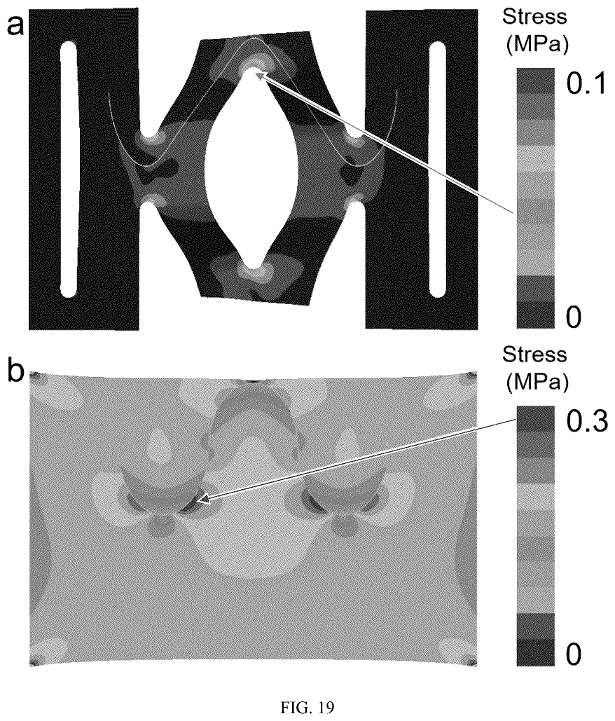

Finite element analysis (FEA) on certain embodiments of BCEMs confirmed the strain tolerance of the devices. Under 25% of elongation, the deformation of kirigami structures accommodate the macroscopic stretching, and lead to only about 30 kPa of stress in the serpentine interconnect (FIG. 2(g)), which is well below the failure threshold for the constituent materials. In contrast, the membrane without kirigami would lead to stresses that are about 2 times higher than those in kirigami-structured membrane. Further details of FEA including mesh generation and stress distribution of substrates are demonstrated in FIG. 18 and FIG. 19. Indeed, the reduced stress levels associated with kirigami patterning are helpful for the inhibition of mechanical failure of electrical circuits and interfacial delamination. The deformability of certain embodiments of BCEMs can be tuned with variations of the cutting pattern and corresponding changes of the serpentine patterns. In certain embodiments, ultimate stretchability scales with

$$\frac{{L}_{c} - x}{2 y} ,$$

where L_{c} is the length of the cut, x is the spacing between nearest cuts in the transverse direction, and y is the spacing in the axial direction.³¹ In an alternative design, the stretchability of the electronic components can increase from about 120% to about 350%, as y is reduced from 5 mm to 2 mm and x is reduced from 4 mm to 2 mm (FIG. 2(f) and FIG. 10(b)).

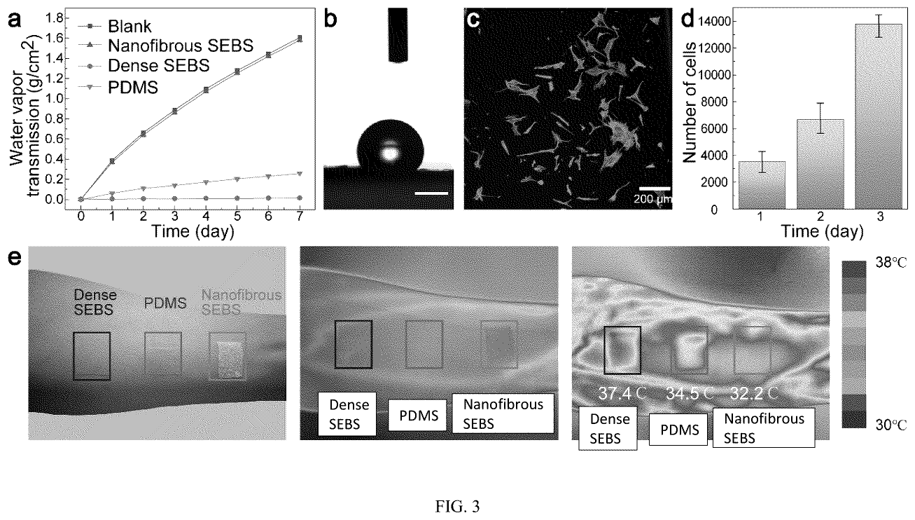

The BCEMs of certain embodiments exhibit good breathability and biocompatibility for skin-integration. Water vapor transmission rate (WVTR) serves as a key indicator of the breathability of membranes, and it is determined by measuring the weight loss of water-filled containers sealed with various membranes of interest. 35 The testing procedures were based on ASTM E96, and showed WVTR of a continuous SEBS nanofibrous membrane without kirigami cuts (FIG. 3(a)) is eight times higher than that of a 100-μm-thick polydimethylsiloxane (PDMS) film, which represents a common substrate for other skin-integrated soft electronics. The WVTR associated with nanofibrous substrates is similar to that of an open container without cover. In addition, the small spacing between open channels (e.g., spacing <1 μm) in the nanofibrous membranes is unlikely to cause blockage of natural sweat pores, which is advantageous over perforated dense substrates with a typical spacing between pores on the order of ˜100 μm. The high vapor permeability of the substrate involved in certain embodiments of BCEMs is advantageous for skin application, since sweat evaporation is essential for user comfort and the inhibition of inflammation caused by perspiration accumulation. The inventors performed on-skin tests to further demonstrate the breathability of the nanofibrous substrate (FIG. 3(e)). Three films involving dense PDMS, dense SEBS, and nanofibrous SEBS were separately attached to the forearm of a healthy volunteer during a 30-minute running exercise. An infrared camera was used to measure the skin temperatures before and after the exercise. In contrast to the dense films, the nanofibrous membrane induced little variation of the skin temperature during the exercise, indicating effective cooling by sweat evaporation (e.g., embodiments have been shown to provide a difference as high as 5 degrees C. when compared to related art, and less than 1 degree C. difference, alternatively less than 0.5 degrees C., alternatively less than about 0.1 degrees C., alternatively not detectable in a thermal image having 10 color gradations between 30 degrees C. and 38 degrees C., between adjacent uncovered skin and embodiments with nanofibrous SEBS membrane as shown, for example, in FIG. 3(e)). The hydrophobicity of SEBS was enhanced with the nanofibrous surface topography (FIG. 3(b) and FIG. 13), which helps to inhibit capillary forces that could damage the device. Biocompatibility of BCEMs were confirmed with cell culture experiments. Live/Dead assay on NIH 3T3 fibroblasts cultured on a representative device shows good viability and morphology of the cells (FIG. 3(c)). In addition, cell proliferation over 3 days of incubation was quantified with MTT (3-[4,5-dimethylthiazol-2-yl]-2,5 diphenyl tetrazolium bromide) assay (FIG. 3(d)).

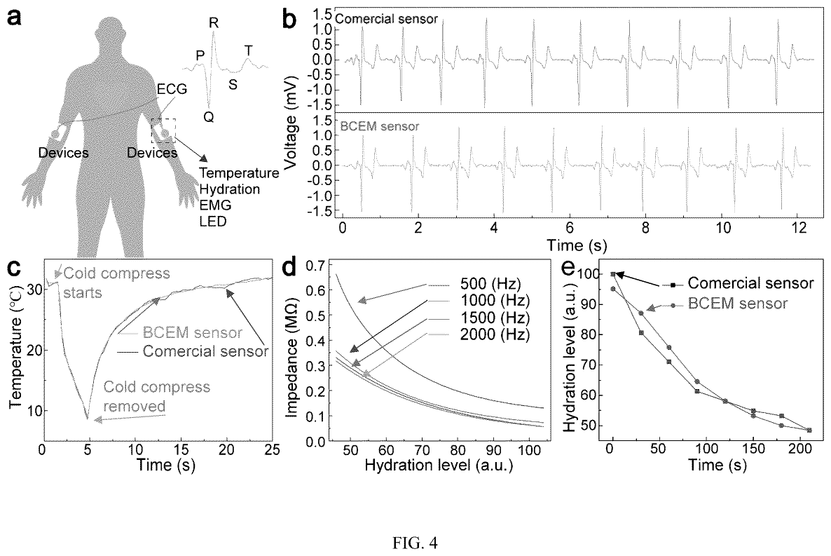

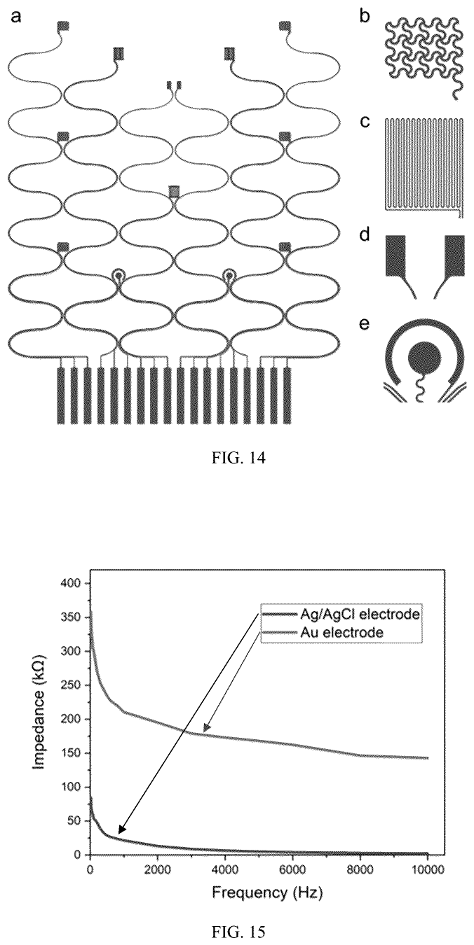

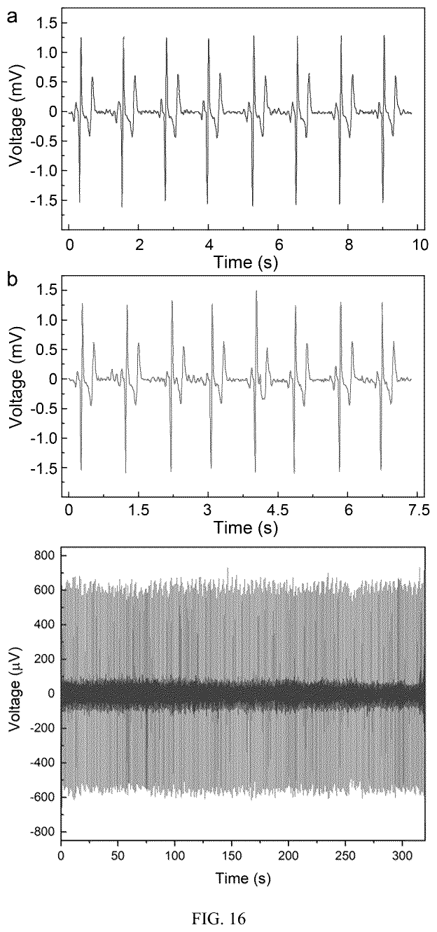

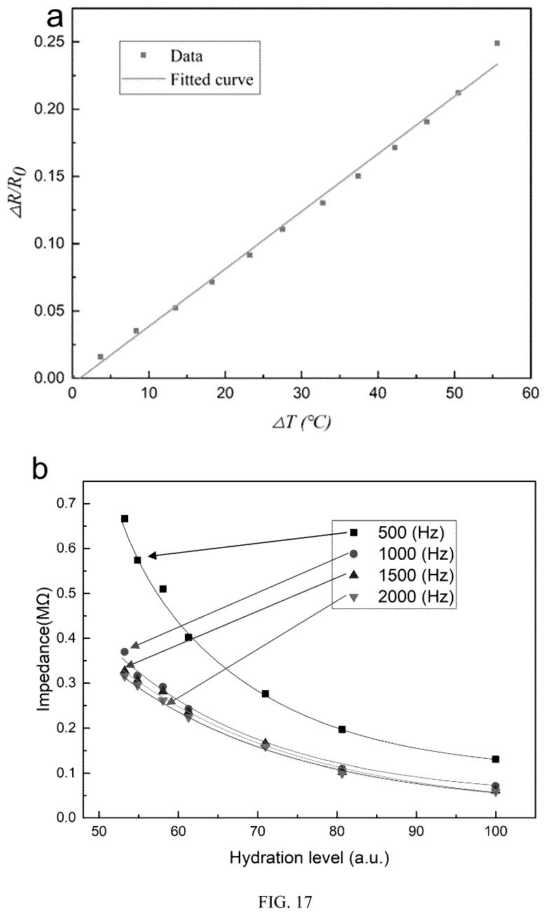

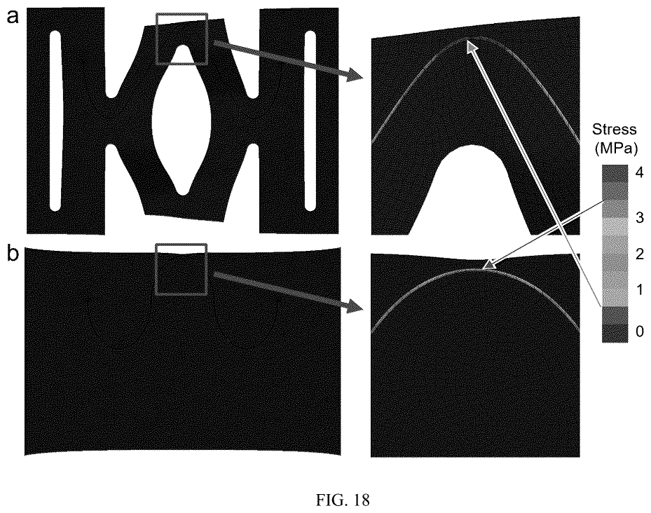

A representative BCEM according to an embodiment of the subject invention can provide 6 unipolar electrodes, 3 temperature sensors, 2 hydration sensors, and one pair of bipolar electrodes for further integration with semiconductor optoelectronics (FIG. 14). Such an array of sensors enables multi-modal physiological measurement on the skin. ECG signals were obtained from two BCEMs separately mounted on the left and right forearms of a healthy volunteer (FIG. 4(a)). The contact impedance between the BCEM electrode and the skin is about 300 kΩ at 100 Hz (FIG. 15), which is sufficient for gathering high-quality electrophysiological signals comparable to those obtained with commercial silver/silver chloride (Ag/AgCl) gel electrodes (FIG. 4(b)). The ECG signals obtained with BCEMs were stable after doing exercises or over a continuous period of measurement (FIG. 16). The temperature sensors in certain embodiments of BCEMs consist of serpentine traces of gold (Au, 20 μm in width and 30 nm in thickness), where changes in resistance correlate to the changes in temperature. The temperature coefficient of resistance for the sensors in this embodiment was 4.3×10⁻³° C.⁻¹ in physiologically relevant range (FIG. 17(a)). The functionality of temperature sensors was examined with cold compress applied on top of the skin where the sensor was laminated. The BCEM captured the temperature drop associated with the cold compress and the recovery to the baseline as the cold compress was removed. The temperature measured with BCEM is consistent with the results measured by a commercial thermocouple (FIG. 4(c)). The hydration sensor involves coaxial dot-ring Au electrodes, which characterizes skin hydration through the measurement of electrical impedance. A commercial skin hydration sensor was used for the calibration for BCEMs in FIG. 17(b). FIG. 4(d) demonstrates the impedance captured by an embodiment of a BCEM as a function of skin hydration levels, measured at various frequencies. In another experiment, the natural attenuation of skin hydration levels after applying moisturizing lotion were measured with both commercial sensors and BCEM sensors. The hydration responses recorded by the two types of sensors are consistent, demonstrating the stable performance of this embodiment of the BCEM (FIG. 4(e)).

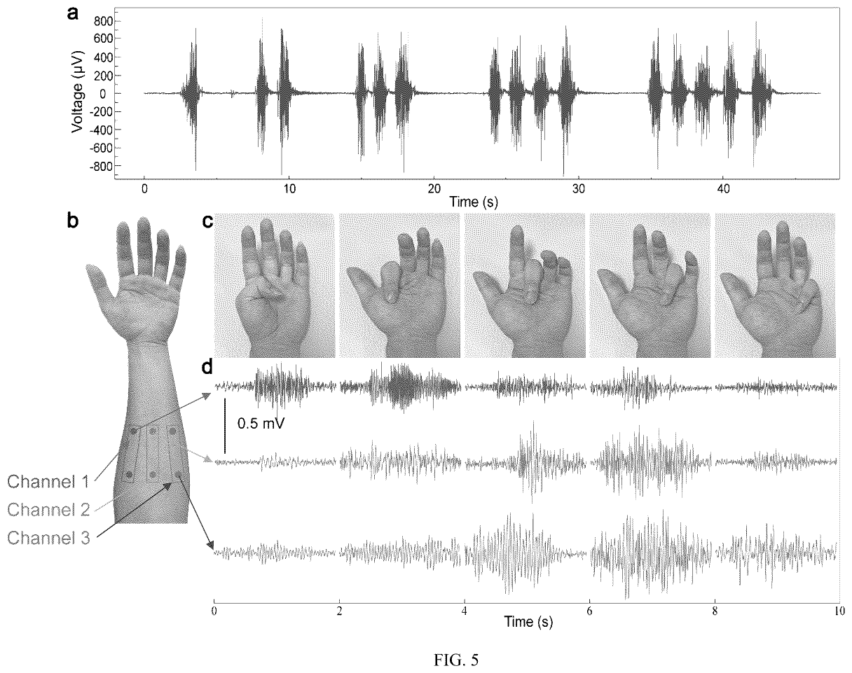

EMG signals generated from the contraction of muscles are essential for medical diagnosis, control of prosthesis, human-robot interactions, virtual/augmented reality, and other technologies. The inventors exploited BCEMs for EMG-based gesture recognition to demonstrate their potential for further applications. The large-area and conformal contact between BCEMs and the skin surface, along with their good breathability, can be advantageous for spatiotemporal recording of EMG. Continuous measurement with BCEM electrodes generated EMG recordings with high signal quality (FIG. 5(a)). To differentiate various gestures, the inventors chose three pairs of electrodes mounted across the forearm of a healthy volunteer and obtained simultaneous recordings from these three independent channels (FIG. 5(b)). The five different modes of motion are distinguishable with the distinct activation patterns of EMG from the corresponding muscle groups (FIG. 5(c)).

### CONCLUSION

Embodiments of the subject invention provide a route for hybrid integration of high-performance microsensors and nanofibrous substrates for the construction of breathable and skin-conformal electronics. The fabrication techniques employed and tested in accordance with specific embodiments have been shown to mitigate the mismatches in processing conditions for various device components, allowing for scalable manufacturing of multifunctional devices tailored for skin-integration. The devices constructed with these techniques can combine excellent sensory performance with breathability and conformability to the skin surfaces, which is advantageous for extended use as wearable systems. Embodiments have demonstrated realistic applications involving spatiotemporal measurement of temperature, hydration, ECG, and EMG, and could support additional functionalities built on this platform due to the versatility of planar microfabrication and transfer printing methods. Use of semiconductor electronics, biochemical sensors, and micro-actuators for integration on these devices will expand their utility, creating advanced tools for disease management, human-machine interface, and other applications.

Materials and Methods

### Device Fabrication

Fabrication of BCEMs began with spin coating a thin layer of poly (methyl methacrylate) (PMMA) (Sigma-Aldrich, MW 350k) on a 4-inch silicon wafer, which serves as the sacrificial layer. An encapsulation layer of polyimide (PI) (Sigma-Aldrich, product #575801) was spin-coated and cured on top of PMMA. Next, a bilayer of chromium (Cr, 5 nm) and gold (Au, 50 nm) was sputtered (Denton Desktop Pro) on the PI layer, followed by photolithography (AZ 5214 and URE-2000/35L) and wet etching to obtain the 1^{st} metallic layer with a width of 20 μm, serving as the temperature sensing element. The 2^{nd} metallic layer consisting of Cr (5 nm) and Au (200 nm) was deposited and patterned for electrodes, hydration sensors and interconnects. Another PI encapsulation layer was applied on top of the sensor components, and the overall structures of the serpentine devices were defined with reactive ion etching (RIE, Tailong Electronics) with oxygen-plasma, which completed the microfabrication process.

Water-soluble tapes (3M) were used to pick up the planar fabricated microdevices after dissolving PMMA in acetone for 12 h. SEBS powders (H1062; Asahi Kasei) were dissolved in chloroform/toluene (8:2) to form a 15 wt % SEBS solution. The tape-supported microsensors were anchored on the rolling substrate of an electrospinning equipment (Beijing Yongkang Co. Ltd.) to manufacture SEBS nanofibrous membranes. After releasing the tape in water, laser cutting was employed to obtain kirigami pattern in BCEMs.

### FEA Simulation

Static analysis was employed to predict the stress distribution in a kirigami sheet bonded with serpentine interconnects using a commercial software package (ABAQUS). The Young's moduli (E) and Poisson's ratio (v) of the materials used in the simulations are E_{interconnects}=100 MPa, and v_{interconnects}=0.4 for interconnects, and E_{substrate}=1 MPa and v_{substrate}=0.3 for substrate. Shell elements were applied to the model to get fine mesh distribution which can achieve the convergence of the non-linear problem. A small perturbation was added in the Z-axis direction, so that the kirigami film can achieve 3D out-of-plane deformation.

### Cell Culture and Biocompatibility

NIH 3T3 fibroblasts were used to examine the in vitro cytotoxicity of BCEMs. After disinfecting using ethyl alcohol, BCEMs were irradiated with UVO to improve surface energy to facilitate the adhesion of fibronectin. BCEMs were soaked in fibronectin for 24 h and then seeded with the cell suspension. After 24 h of incubation, the cells were observed using confocal fluorescent microscope (Nikon, Japan). MTT biocompatibility assay was used to quantify the cell proliferation. The absorption at 540 nm, which reflects the cell metabolic activity, was detected using a plate reader (BioTek, US). All samples were cultured at 37° C. in an incubator with 5% CO₂.

### Characterization and Equipment

Scanning electron microscopy (SEM) images of BCEM were taken with Hitachi S4800. Mechanical characterization and interfacial toughness were carried out with Zwick Roell tensile tester. A digital source meter (2450; Keithley Instruments) is applied to record the variation of resistance upon uniaxial tensile and cyclic testing. The interfacial toughness was determined by dividing the plateau force by the width of the SEBS sheet.³⁶ Hydrophobicity of the membranes was measured with a video water contact angle system (VCA 2500XE; AST Products). Electrophysiological signals were measured using a commercial data acquisition system (PowerLab T26, AD Instruments). Adding a layer of conductive gel (SignaGel Electrode Gel, USA) between electrodes and the skin improved the signal quality for electrophysiological measurements. Impedance/resistance for skin hydration and temperature were recorded with a LCR meter (E4980AL; Keysight Instruments). The calibration for skin hydration sensor was done with a commercial hydrometer (Real Bubee). Thermal images were taken with an infrared imaging camera (Fluke Ti480).

All patents, patent applications, provisional applications, and publications referred to or cited herein are incorporated by reference in their entirety, including all figures and tables, to the extent they are not inconsistent with the explicit teachings of this specification.

In order that the present disclosure may be more readily understood, certain terms are defined below, and throughout the detailed description, to provide guidance as to their meaning as used herein.

As used herein, the terms “a,” “an,” “the” and similar terms used in the context of the present invention are to be construed to cover both the singular and plural unless otherwise indicated herein or clearly contradicted by the context. Thus, for example, reference to “an arm” or “a hole” should be construed to cover or encompass both a singular arm or a singular hole and a plurality of arms and a plurality of holes, unless indicated otherwise or clearly contradicted by the context.

As used herein, the terms “about” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “and/or” should be understood to mean “either or both” of the features so conjoined, i.e., elements that are conjunctively present in some cases and disjunctively present in other cases.

As used herein, the terms “comprising”, “consisting of” and “consisting essentially of” are defined according to their standard meaning. The terms may be substituted for one another herein in order to attach the specific meaning associated with each term.

As used herein, the term “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The present disclosure is to be considered as an exemplification of the invention and is not intended to limit the invention to the specific embodiments illustrated by the figures or description below.

### EXEMPLIFIED EMBODIMENTS

The invention may be better understood by reference to certain illustrative examples, including but not limited to the following:

Embodiment 1. A breathable conformal electronic membrane (BCEM), the BCEM comprising:
a multiplicity of planar-fabricated electronic components arranged in a stretchable serpentine pattern and selectively encapsulated with at least one encapsulating layer; and
a flexible, mass permeable, and porous substrate comprising electrospun polymer nanofibers bonded to the electronic components, the encapsulating layer, or both;
the porous substrate comprising a network of kirigami cuts configured and adapted to mitigate stress concentration during deformation while maintaining connectivity and structural integrity of the electronic components.

Embodiment 2. The BCEM according to Embodiment 1, wherein the BCEM has a non-deformed configuration having a planar surface area of less than 80 square centimeters, and a deformed configuration having a planar surface area equal to or greater than about 200 square centimeters.

Embodiment 3. The BCEM according to Embodiment 2, wherein the BCEM has a non-deformed configuration having a water vapor transmission greater than 0.1 g/cm 2 per day.

Embodiment 4. The BCEM according to Embodiment 3, wherein the planar-fabricated electronic components comprise a multiplicity of microfabricated sensors selected from the group consisting of bipolar electrodes, unipolar electrodes, temperature sensors, and hydration sensors.

Embodiment 5. The BCEM according to Embodiment 4, wherein the planar-fabricated electronic components comprise at least one additional device comprising an inorganic semiconductor, biochemical reagent, or both; the additional device configured and adapted to provide one or more extended functions.

Embodiment 6. The BCEM according to Embodiment 4, wherein the planar-fabricated electronic components comprise a multifunctional sensor array configured and adapted for spatiotemporal measurement of at least one parameter selected from the group consisting of body temperature, electrocardiogram (ECG), skin hydration, and electromyogram (EMG).

Embodiment 7. The BCEM according to Embodiment 4, wherein the network of kirigami cuts is arranged in an offset array comprising a multiplicity of horizontal rows and a multiplicity of vertical columns, each row comprising a multiplicity of aligned horizontal cuts, each cut separated from respective adjacent cuts by a gap, wherein each respective row above a bottom row is offset such that a majority of gaps in each row are vertically aligned above a midpoint of a cut in the row below.

Embodiment 8. The BCEM according to Embodiment 7, wherein each respective cut comprises a common length of cut, L_{c}, each respective gap comprises a common gap length, x, and each respective row above a first row is separated from the row below by a common height, y; such that the array is a regular repeating array arranged such that each respective cut in each respective row above the first two rows is aligned with a respective cut in a second row below and each respective gap in each respective row above the first two rows is aligned with a respective gap in the second row below.

Embodiment 9. The BCEM according to Embodiment 8, wherein the values of L_{c}, x, and y, respectively are selected such that the ratio

$$\frac{( {L}_{c} - x )}{2 y}$$

is equal to or greater than 2.

Embodiment 10. The BCEM according to Embodiment 9, wherein the planar-fabricated electronic components are configured, adapted, and aligned with respective kirigami cuts and respective gaps to produce deformation-invariant electrical performance with less than 10% change in resistance (ΔR/R₀) as the BCEM is exposed to each of (1) stretching uniaxially from 0% to 120%, (2) twisting by 720°, and (3) loading with 10,000 cycles of 80% elongation.

Embodiment 11. A method of making a breathable conformal electronic membrane (BCEM), the method comprising:
fabricating a multiplicity of electronic components connected with or comprising stretchable serpentine patterns on a planar wafer having a planar surface area of less than 80 square centimeters;
selectively encapsulating the electronic components with a polyimide (PI) layer configured and adapted to inhibit crosstalk between components or current leakage;
placement of a sacrificial layer based on poly(methyl methacrylate) (PMMA) between the electronic components and the planar substrate;
releasing the electronic components from the planar wafer after dissolving PMMA in acetone;
picking up the electronic components with a stamp comprising a water-soluble tape to create a device-on-stamp; and
electrospinning polymer on the device-on-stamp to create a porous substrate having a desired flexibility and mass permeability.

Embodiment 12. The method according to Embodiment 11, comprising:
dissolving the water-soluble tape to yield a freestanding membrane device comprising the electronic components and the porous substrate;
laser cutting the porous substrate to incorporate kirigami structures.

Embodiment 13. The method according to Embodiment 12, wherein the cuts are placed in a periodic and alternating pattern configured and adapted to advantageously endow high stretchability of the freestanding membrane device, providing a stretched length of the freestanding membrane device that is more than 250% of an unstretched length of the freestanding membrane device, with conformability on 3D curved surfaces.

Embodiment 14. The method according to Embodiment 13, wherein the kirigami structures are provided round edges during the laser cutting process, the round edges configured and adapted to mitigate stress concentration during deformation.

Embodiment 15. The method according to Embodiment 12, wherein the electronic components comprise one or more of microfabricated bipolar electrodes, unipolar electrodes, temperature sensors, and hydration sensors.

Embodiment 16. The method according to Embodiment 12, comprising:
expanding the electronic components by stretching the kirigami structures of the porous substrate to cover a sensor surface area equal to or greater than about 200 square centimeters.

Embodiment 17. The method according to Embodiment 16, comprising:
attaching an adhesive layer to the porous substrate, the adhesive layer configured and adapted for conformal adhesion to a three dimensional biological surface.

Embodiment 18. The method according to Embodiment 17, the adhesive layer configured and adapted for conformal adhesion to an exterior anatomical region of a human patient.

Embodiment 19. The method according to Embodiment 11, comprising:
creating a multiplicity of device-on-stamps; and
creating a continuous membrane with roll-to-roll processing for electrospinning the polymer onto the multiplicity of the device-on-stamps to create a porous substrate having a desired flexibility and mass permeability.

Embodiment 20. A breathable conformal electronic membrane (BCEM), the BCEM comprising:
a multiplicity of planar-fabricated electronic components arranged in a stretchable serpentine pattern and selectively encapsulated with at least one encapsulating layer; and
a flexible, mass permeable, and porous substrate comprising electrospun polymer nanofibers bonded to the planar-fabricated electronic components, the encapsulating layer, or both;
the porous substrate comprising a network of kirigami cuts configured and adapted to mitigate stress concentration during deformation while maintaining connectivity and structural integrity of the planar-fabricated electronic components;
wherein the BCEM has a non-deformed configuration having a planar surface area of less than 80 square centimeters, and a deformed configuration having a planar surface area equal to or greater than about 200 square centimeters,
wherein the BCEM in the non-deformed configuration has a water vapor transmission greater than 0.1 g/cm 2 per day,
wherein the planar-fabricated electronic components comprise a multiplicity of microfabricated sensors selected from the group consisting of bipolar electrodes, unipolar electrodes, temperature sensors, and hydration sensors;
wherein the network of kirigami cuts is arranged in an offset array comprising a multiplicity of horizontal rows and a multiplicity of vertical columns, each row comprising a multiplicity of aligned horizontal cuts, each cut separated from respective adjacent cuts by a gap, wherein each respective row above a bottom row is offset such that a majority of gaps in each row are vertically aligned above a midpoint of a cut in the row below;
wherein each respective cut comprises a common length of cut, L_{c}, each respective gap comprises a common gap length, x, and each respective row above a first row is separated from the row below by a common height, y; such that the array is a regular repeating array arranged such that each respective cut in each respective row above the first two rows is aligned with a respective cut in the second row below and each respective gap in each respective row above the first two rows is aligned with a respective gap in the second row below;
wherein the values of L_{c}, x, and y, respectively are selected such that the ratio

$$\frac{( {L}_{c} - x )}{2 y}$$

is equal to or greater than 2; and
wherein the planar-fabricated electronic components are configured, adapted, and aligned with respective kirigami cuts and respective gaps to produce deformation-invariant electrical performance with less than 10% change in resistance (ΔR/R₀) as the BCEM is exposed to each of (1) stretching uniaxially from 0% to 120%, (2) twisting by 720°, and (3) loading with 10,000 cycles of 80% elongation.

Although the present invention has been illustrated and described herein with reference to preferred embodiments and specific examples thereof, it will be readily apparent to those of ordinary skill in the art that other embodiments and examples may perform similar functions and/or achieve like results.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

(1) Wang, C.; Wang, C.; Huang, Z.; Xu, S. Materials and Structures toward Soft Electronics. Adv. Mater. 2018, 30, 1801368.
(2) Li, H.; Liu, H.; Sun, M.; Huang, Y.; Xu, L. 3D Interfacing between Soft Electronic Tools and Complex Biological Tissues. Adv. Mater. 2020, 33, 2004425.
(3) Zhang, Y.; Zhang, T.; Huang, Z.; Yang, J. A New Class of Electronic Devices Based on Flexible Porous Substrates. Advanced Science. 2022, 2105084.
(4) Jung, D.; Lim, C.; Shim, H. J.; Kim, Y.; Park, C.; Jung, J.; Han, S. I.; Sunwoo, S.; Cho, K. W.; Cha, G. D.; Kim, D. C.; Koo, J. H.; Kim, J. H.; Hyeon, T.; Kim, D. Highly Conductive and Elastic Nanomembrane for Skin Electronics. Science. 2021, 373, 1022-1026.
(5) Guo, S.; Wu, K.; Guo, S.; Wu, K.; Li, C.; Wang, H.; Sun, Z.; Xi, D.; Zhang, S. Article Integrated Contact Lens Sensor System Based on Multifunctional Ultrathin MoS 2 Transistors Integrated Contact Lens Sensor System Based on Multifunctional Ultrathin MoS 2 Transistors. Matter 2021, 4, 969-985.
(6) Zhu, Z.; Guo, S.-Z.; Hirdler, T.; Eide, C.; Fan, X.; Tolar, J.; McAlpine, M. C. 3D Printed Functional and Biological Materials on Moving Freeform Surfaces. Adv. Mater. 2018, 30, 1707495.
(7) Hong, S.; Zagni, N.; Choo, S.; Liu, N.; Baek, S.; Bala, A.; Yoo, H.; Kang, B. H.; Kim, H. J.; Yun, H. J.; Alam, M. A.; Kim, S. Highly Sensitive Active Pixel Image Sensor Array Driven by Large-Area Bilayer MoS2 Transistor Circuitry. Nat. Commun. 2021, 12, 3559.
(8) Jun, J. J.; Steinmetz, N. A.; Siegle, J. H.; Denman, D. J.; Bauza, M.; Barbarits, B.; Lee, A. K.; Anastassiou, C. A.; Andrei, A.; Aydin, C.; Barbic, M.; Blanche, T. J.; Bonin, V.; Couto, J.; Dutta, B.; Gratiy, S. L.; Gutnisky, D. A.; Musser, M.; Karsh, B.; Ledochowitsch, P.; Lopez, C. M.; Mitelut, C.; Musa, S.; Okun, M.; Pachitariu, M.; Putzeys, J.; Rich, P. D.; Rossant, C.; Sun, W. L.; Svoboda, K.; Carandini, M.; Harris, K. D.; Koch, C.; O'Keefe, J.; Harris, T. D. Fully Integrated Silicon Probes for High-Density Recording of Neural Activity. Nature 2017, 551, 232-236.
(9) Huang, Z.; Hao, Y.; Li, Y.; Hu, H.; Wang, C.; Nomoto, A.; Pan, T.; Gu, Y.; Chen, Y.; Zhang, T.; Li, W.; Lei, Y.; Kim, N. H.; Wang, C.; Zhang, L.; Ward, J. W.; Maralani, A.; Li, X.; Durstock, M. F.; Pisano, A.; Lin, Y.; Xu, S. Three-Dimensional Integrated Stretchable Electronics. Nat. Electron. 2018, 1, 473-480.
(10) Lee, H.; Song, C.; Hong, Y. S.; Kim, M. S.; Cho, H. R.; Kang, T.; Shin, K.; Choi, S. H.; Hyeon, T.; Kim, D. Wearable/Disposable Sweat-Based Glucose Monitoring Device with Multistage Transdermal Drug Delivery Module. Sci. Adv. 2017, 3, 1-9.
(11) Rao, Z.; Lu, Y.; Li, Z.; Sim, K.; Ma, Z.; Xiao, J.; Yu, C. Curvy, Shape-Adaptive Imagers Based on Printed Optoelectronic Pixels with a Kirigami Design. Nat. Electron. 2021, 4, 513-521.
(12) Wang, L.; Fu, X.; He, J.; Shi, X.; Chen, T.; Chen, P.; Wang, B.; Peng, H. Application Challenges in Fiber and Textile Electronics. Adv. Mater. 2020, 32, 1901971.
(13) Chen, G.; Xiao, X.; Zhao, X.; Tat, T.; Bick, M.; Chen, J. Electronic Textiles for Wearable Point-of-Care Systems. Chem. Rev. 2022, 122, 3259-3291.
(14) Jang, K. I.; Han, S. Y.; Xu, S.; Mathewson, K. E.; Zhang, Y.; Jeong, J. W.; Kim, G. T.; Webb, R. C.; Lee, J. W.; Dawidczyk, T. J.; Kim, R. H.; Song, Y. M.; Yeo, W. H.; Kim, S.; Cheng, H.; Rhee, S. Il; Chung, J.; Kim, B.; Chung, H. U.; Lee, D.; Yang, Y.; Cho, M.; Gaspar, J. G.; Carbonari, R.; Fabiani, M.; Gratton, G.; Huang, Y.; Rogers, J. A. Rugged and Breathable Forms of Stretchable Electronics with Adherent Composite Substrates for Transcutaneous Monitoring. Nat. Commun. 2014, 5, 1-10.
(15) Tan, C.; Dong, Z.; Li, Y.; Zhao, H.; Huang, X.; Zhou, Z.; Jiang, J.-W.; Long, Y.-Z.; Jiang, P.; Zhang, T.-Y. A High Performance Wearable Strain Sensor with Advanced Thermal Management for Motion Monitoring. Nat. Commun. 2020, 11, 1-10.
(16) Li, Z.; Zhu, M.; Shen, J.; Qiu, Q.; Yu, J.; Ding, B. All-fiber Structured Electronic Skin with High Elasticity and Breathability. Adv. Funct. Mater. 2019, 30, 1908411.
(17) Guo, H.; Tan, Y. J.; Chen, G.; Wang, Z.; Susanto, G. J.; See, H. H.; Yang, Z.; Lim, Z. W.; Yang, L.; Tee, B. C. K. Artificially Innervated Self-Healing Foams as Synthetic Piezo-Impedance Sensor Skins. Nat. Commun. 2020, 11, 1-10.
(18) Visser, C. W.; Amato, D. N.; Mueller, J.; Lewis, J. A. Architected Polymer Foams via Direct Bubble Writing. Adv. Mater. 2019, 31, 1904668.
(19) Fu, Q.; Chen, Y.; Sorieul, M. Wood-Based Flexible Electronics. ACS Nano 2020, 14, 3528-3538.
(20) Zhu, H.; Luo, W.; Ciesielski, P. N.; Fang, Z.; Zhu, J. Y.; Henriksson, G.; Himmel, M. E.; Hu, L. Wood-Derived Materials for Green Electronics, Biological Devices, and Energy Applications. Chem. Rev. 2016, 116, 9305-9374.
(21) Zou, B.; Chen, Y.; Liu, Y.; Xie, R.; Du, Q.; Zhang, T.; Shen, Y.; Zheng, B.; Li, S.; Wu, J. Repurposed Leather with Sensing Capabilities for Multifunctional Electronic Skin. Adv. Sci. 2019, 6, 1801283.
(22) Wang, L.; Wang, L.; Zhang, Y.; Pan, J.; Li, S.; Sun, X.; Zhang, B.; Peng, H. Weaving Sensing Fibers into Electrochemical Fabric for Real-time Health Monitoring. Adv. Funct. Mater. 2018, 28, 1804456.
(23) Fan, Y. J.; Li, X.; Kuang, S. Y.; Zhang, L.; Chen, Y. H.; Liu, L.; Zhang, K.; Ma, S. W.; Liang, F.; Wu, T. Highly Robust, Transparent, and Breathable Epidermal Electrode. ACS Nano 2018, 12, 9326-9332.
(24) Xu, Y.; Sun, B.; Ling, Y.; Fei, Q.; Chen, Z.; Li, X.; Guo, P.; Jeon, N.; Goswami, S.; Liao, Y.; Ding, S.; Yu, Q.; Lin, J.; Huang, G.; Yan, Z. Multiscale Porous Elastomer Substrates for Multifunctional On-Skin Electronics with Passive-Cooling Capabilities. Proc. Natl. Acad. Sci. U.S.A 2020, 117, 205-213.
(25) Ma, Z.; Huang, Q.; Xu, Q.; Zhuang, Q.; Zhao, X.; Yang, Y.; Qiu, H.; Yang, Z.; Wang, C.; Chai, Y.; Zheng, Z. Permeable Superelastic Liquid-Metal Fibre Mat Enables Biocompatible and Monolithic Stretchable Electronics. Nat. Mater. 2021, 20, 859-868.
(26) Dickey, M. D. Stretchable and Soft Electronics Using Liquid Metals. Advanced Materials. 2017, 1606425.
(27) Qi, K.; He, J.; Wang, H.; Zhou, Y.; You, X.; Nan, N.; Shao, W.; Wang, L.; Ding, B.; Cui, S. A Highly Stretchable Nanofiber-Based Electronic Skin with Pressure-, Strain-, and Flexion-Sensitive Properties for Health and Motion Monitoring. ACS Appl. Mater. Interfaces 2017, 9, 42951-42960.
(28) Lu, C.; Chen, X. Electrospun Polyaniline Nanofiber Networks toward High-performance Flexible Supercapacitors. Adv. Mater. Technol. 2019, 4, 1900564.
(29) Matsuhisa, N.; Inoue, D.; Zalar, P.; Jin, H.; Matsuba, Y.; Itoh, A.; Yokota, T.; Hashizume, D.; Someya, T. Printable Elastic Conductors by in Situ Formation of Silver Nanoparticles from Silver Flakes. Nat. Mater. 2017, 16, 834-840.
(30) Wang, Y.; Yin, L.; Bai, Y.; Liu, S.; Wang, L.; Zhou, Y.; Hou, C.; Yang, Z.; Wu, H.; Ma, J.; Shen, Y.; Deng, P.; Zhang, S.; Duan, T.; Li, Z.; Ren, J.; Xiao, L.; Yin, Z.; Lu, N.; Huang, Y. A. Electrically Compensated, Tattoo-like Electrodes for Epidermal Electrophysiology at Scale. Sci. Adv. 2020, 6, eabd0996.
(31) Shyu, T. C.; Damasceno, P. F.; Dodd, P. M.; Lamoureux, A.; Xu, L.; Shlian, M.; Shtein, M.; Glotzer, S. C.; Kotov, N. A. A Kirigami Approach to Engineering Elasticity in Nanocomposites through Patterned Defects. Nat. Mater. 2015, 14, 785-789.
(32) Zhao, R.; Lin, S.; Yuk, H.; Zhao, X. Kirigami Enhances Film Adhesion. Soft Matter 2018, 14, 2515-2525.
(33) Kim, D. H.; Lu, N.; Ma, R.; Kim, Y. S.; Kim, R. H.; Wang, S.; Wu, J.; Won, S. M.; Tao, H.; Islam, A.; Yu, K. J.; Kim, T. Il; Chowdhury, R.; Ying, M.; Xu, L.; Li, M.; Chung, H. J.; Keum, H.; McCormick, M.; Liu, P.; Zhang, Y. W.; Omenetto, F. G.; Huang, Y.; Coleman, T.; Rogers, J. A. Epidermal Electronics. Science. 2011, 333, 838-843.
(34) Dagdeviren, C.; Shi, Y.; Joe, P.; Ghaffari, R.; Balooch, G.; Usgaonkar, K.; Gur, O.; Tran, P. L.; Crosby, J. R.; Meyer, M.; Su, Y.; Webb, R. C.; Tedesco, A. S.; Slepian, M. J.; Huang, Y.; Rogers, J. A. Conformal Piezoelectric Systems for Clinical and Experimental Characterization of Soft Tissue Biomechanics. Nat. Mater. 2015, 14, 728-736.
(35) Sun, B.; McCay, R. N.; Goswami, S.; Xu, Y.; Zhang, C.; Ling, Y.; Lin, J.; Yan, Z. Gas-permeable, Multifunctional On-skin Electronics Based on Laser-induced Porous Graphene and Sugar-templated Elastomer Sponges. Adv. Mater. 2018, 30, 1804327.
(36) Yuk, H.; Zhang, T.; Lin, S.; Parada, G. A.; Zhao, X. Tough Bonding of Hydrogels to Diverse Non-Porous Surfaces. Nat. Mater. 2016, 15, 190-196.

We claim:

1. A breathable conformal electronic membrane (BCEM), the BCEM comprising:
a multiplicity of planar-fabricated electronic components arranged in a stretchable serpentine pattern and selectively encapsulated with at least one encapsulating layer; and
a flexible, mass permeable, and porous substrate comprising electrospun polymer nanofibers bonded to the electronic components, the encapsulating layer, or both;
the porous substrate comprising a network of kirigami cuts configured and adapted to mitigate stress concentration during deformation while maintaining connectivity and structural integrity of the electronic components.

2. The BCEM according to claim 1, wherein the BCEM has a non-deformed configuration having a planar surface area of less than 80 square centimeters, and a deformed configuration having a planar surface area equal to or greater than about 200 square centimeters.

3. The BCEM according to claim 2, wherein the BCEM has a non-deformed configuration having a water vapor transmission greater than 0.1 g/cm² per day.

4. The BCEM according to claim 3, wherein the planar-fabricated electronic components comprise a multiplicity of microfabricated sensors selected from the group consisting of bipolar electrodes, unipolar electrodes, temperature sensors, and hydration sensors.

5. The BCEM according to claim 4, wherein the planar-fabricated electronic components comprise at least one additional device comprising an inorganic semiconductor, biochemical reagent, or both; the additional device configured and adapted to provide one or more extended functions.

6. The BCEM according to claim 4, wherein the planar-fabricated electronic components comprise a multifunctional sensor array configured and adapted for spatiotemporal measurement of at least one parameter selected from the group consisting of body temperature, electrocardiogram (ECG), skin hydration, and electromyogram (EMG).

7. The BCEM according to claim 4, wherein the network of kirigami cuts is arranged in an offset array comprising a multiplicity of horizontal rows and a multiplicity of vertical columns, each row comprising a multiplicity of aligned horizontal cuts, each cut separated from respective adjacent cuts by a gap, wherein each respective row above a bottom row is offset such that a majority of gaps in each row are vertically aligned above a midpoint of a cut in the row below.

8. The BCEM according to claim 7, wherein each respective cut comprises a common length of cut, L_{c}, each respective gap comprises a common gap length, x, and each respective row above a first row is separated from the row below by a common height, y; such that the array is a regular repeating array arranged such that each respective cut in each respective row above the first two rows is aligned with a respective cut in a second row below and each respective gap in each respective row above the first two rows is aligned with a respective gap in the second row below.

9. The BCEM according to claim 8, wherein the values of L_{c}, x, and y, respectively are selected such that the ratio
$$\frac{( {L}_{c} - x )}{2 y}$$
is equal to or greater than 2.

10. The BCEM according to claim 9, wherein the planar-fabricated electronic components are configured, adapted, and aligned with respective kirigami cuts and respective gaps to produce deformation-invariant electrical performance with less than 10% change in resistance (ΔR/R₀) as the BCEM is exposed to each of (1) stretching uniaxially from 0% to 120%, (2) twisting by 720°, and (3) loading with 10,000 cycles of 80% elongation.

11. A method of making a breathable conformal electronic membrane (BCEM), the method comprising:
fabricating a multiplicity of electronic components connected with or comprising stretchable serpentine patterns on a planar wafer having a planar surface area of less than 80 square centimeters;
selectively encapsulating the electronic components with a polyimide (PI) layer configured and adapted to inhibit crosstalk between components or current leakage;
placing a sacrificial layer based on poly(methyl methacrylate) (PMMA) between the electronic components and the planar substrate;
releasing the electronic components from the planar wafer after dissolving PMMA in acetone;
picking up the electronic components with a stamp comprising a water-soluble tape to create a device-on-stamp; and
electrospinning polymer on the device-on-stamp to create a porous substrate having a desired flexibility and mass permeability.

12. The method according to claim 11, comprising:
dissolving the water-soluble tape to yield a freestanding membrane device comprising the electronic components and the porous substrate;
laser cutting the porous substrate to incorporate kirigami structures.

13. The method according to claim 12, wherein the laser cuts are placed in a periodic and alternating pattern configured and adapted to advantageously endow high stretchability of the freestanding membrane device, providing a stretched length of the freestanding membrane device that is more than 250% of an unstretched length of the freestanding membrane device, with conformability on 3D curved surfaces.

14. The method according to claim 13, wherein the kirigami structures are provided round edges during the laser cutting process, the round edges configured and adapted to mitigate stress concentration during deformation.

15. The method according to claim 12, wherein the electronic components comprise one or more of microfabricated bipolar electrodes, unipolar electrodes, temperature sensors, and hydration sensors.

16. The method according to claim 12, comprising:
expanding the electronic components by stretching the kirigami structures of the porous substrate to cover a sensor surface area equal to or greater than about 200 square centimeters.

17. The method according to claim 16, comprising:
attaching an adhesive layer to the porous substrate, the adhesive layer configured and adapted for conformal adhesion to a three dimensional biological surface.

18. The method according to claim 17, the adhesive layer configured and adapted for conformal adhesion to an exterior anatomical region of a human patient.

19. The method according to claim 11, comprising:
creating a multiplicity of device-on-stamps; and
creating a continuous membrane with roll-to-roll processing for electrospinning the polymer onto the multiplicity of the device-on-stamps to create a porous substrate having a desired flexibility and mass permeability.

20. A breathable conformal electronic membrane (BCEM), the BCEM comprising:
a multiplicity of planar-fabricated electronic components arranged in a stretchable serpentine pattern and selectively encapsulated with at least one encapsulating layer; and
a flexible, mass permeable, and porous substrate comprising electrospun polymer nanofibers bonded to the planar-fabricated electronic components, the encapsulating layer, or both;
the porous substrate comprising a network of kirigami cuts configured and adapted to mitigate stress concentration during deformation while maintaining connectivity and structural integrity of the planar-fabricated electronic components;
wherein the BCEM has a non-deformed configuration having a planar surface area of less than 80 square centimeters, and a deformed configuration having a planar surface area equal to or greater than about 200 square centimeters,
wherein the BCEM in the non-deformed configuration has a water vapor transmission greater than 0.1 g/cm² per day,
wherein the planar-fabricated electronic components comprise a multiplicity of microfabricated sensors selected from the group consisting of bipolar electrodes, unipolar electrodes, temperature sensors, and hydration sensors;
wherein the network of kirigami cuts is arranged in an offset array comprising a multiplicity of horizontal rows and a multiplicity of vertical columns, each row comprising a multiplicity of aligned horizontal cuts, each cut separated from respective adjacent cuts by a gap, wherein each respective row above a bottom row is offset such that a majority of gaps in each row are vertically aligned above a midpoint of a cut in the row below;
wherein each respective cut comprises a common length of cut, L_{c}, each respective gap comprises a common gap length, x, and each respective row above a first row is separated from the row below by a common height, y; such that the array is a regular repeating array arranged such that each respective cut in each respective row above the first two rows is aligned with a respective cut in the second row below and each respective gap in each respective row above the first two rows is aligned with a respective gap in the second row below;
wherein the values of L_{c}, x, and y, respectively are selected such that the ratio
$$\frac{( {L}_{c} - x )}{2 y}$$
is equal to or greater than 2; and
wherein the planar-fabricated electronic components are configured, adapted, and aligned with respective kirigami cuts and respective gaps to produce deformation-invariant electrical performance with less than 10% change in resistance (ΔR/R₀) as the BCEM is exposed to each of (1) stretching uniaxially from 0% to 120%, (2) twisting by 720°, and (3) loading with 10,000 cycles of 80% elongation.
